(19) United States
(12) Patent Application Publication
(10) Pub. No.: US 2002/0137066 A1
(43) Pub. Date: Sep. 26, 2002

(54) Polynucleotide assay apparatus and polynucleotide assay method

(75) Inventors: Tomoharu Kajiyama, Kokubunji (JP); Yuji Miyahara, Kodaira (JP); Hiroyuki Tomita, Tachikawa (JP); Kazunori Okano, Shiki (JP)

Correspondence Address: MATTINGLY, STANGER & MALUR, P.C., Suite 370, 1800 Diagonal Road, Alexandria, VA, 22314, US

(73) Assignee: Hitachi, Ltd.

(21) Appl. No.: 09/964,608

(22) Filed: Sep. 28, 2001

Related U.S. Application Data
(62) Division of application No. 09/720,522, filed on Dec. 26, 2000.

(51) Int. Cl. C12Q 1/68; C12M 1/34; G01F 1/64

(52) U.S. Cl. 435/6; 435/287.2; 205/777.5

(57) ABSTRACT

A polynucleotide detecting cell provided with a first electrode (111) to which different DNA probes (13, 14, 15, 16) are fixed in luminous areas (3, 4, 5, 6) differing with the type of DNA probe and a second electrode (113-1, 113-2) opposite to the first electrode is used; target polynucleotides are trapped through hybridization of DNA probes fixed to luminous areas with target polynucleotides; an extending reaction is carried out using an ECL-labeled base (dNTP) to extend the hybridized DNA probes; ECL generated by the application of a voltage between the first electrode and the second electrode is detected; and the presence or absence of any extended chain generated by the extending reaction is detected. The DNA detecting cell of simple apparatus configuration and the assay apparatus using it according to the invention are capable of high speed detection of hybridization between target DNA fragments and DNA probes, and a large volume of probe assaying can be accomplished in a short period of time.

### TECHNICAL FIELD

The present invention relates to a polynucleotide detecting cell for detecting DNA, mRNA and the like and carrying out assays on that basis, and an assay apparatus and an assay method using it.

### BACKGROUND ART

There is known a technique whereby a DNA detecting cell to which 16000 probes are fixed is used to hybridize fluorescent-labeled target DNAs and the probes, the fluorescent labels are excited by scanning the whole area of the DNA detecting cell for not more than 15 minutes using a confocal microscope, and the resultant fluorescence is detected to detect the presence of hybrids (Nature Biotechnology 14, 1675-1680, (1996)).

There is a report on a probing method using a DNA probe labeled with electrochemiluminescence (hereinafter abbreviated to ECL) by which a sample DNA is trapped in a bead with a biotin-avidin bond resulting from the qualification of the sample DNA with a biotin group, a DNA probe having a known ECL-labeled base sequence is hybridized with the sample DNA, and the ECL on the bead surface is detected to check the presence or absence of hybridization (Clinical Chemistry, 37, No. 9, 1626-1632 (1991)).

Nucleotides labeled with ECL and oligos labeled with ECL are known (Japanese Translation of Unexamined PCT Application No. Hei 9-505464) are known. Incidentally, various complexes for use in ECL reactions are extensively known (Clinical Chemistry 37, No. 9, 1534-1539 (1991)), J. Electrochem. Soc., Vol. 132, No.4, 842-849 (1985), Japanese Published Unexamined Patent Application No. Hei 7-173185, and Japanese Published Unexamined Patent Application No. Hei 7-309836).

### DISCLOSURE OF THE INVENTION

According to any of the aforementioned methods of the prior art, as many as 16000 positions to which the probes of the DNA detecting cell are fixed have to be scanned with a laser beam, and accordingly routine application of any of these prior art methods as a diagnostic technique involves the problem of insufficient throughput of hybrid detection.

An object of the present invention is to provide a polynucleotide detecting cell for high speed detection of a hybrid between a target polynucleotide and a probe, and a polynucleotide detecting apparatus and an assay method using it.

In the polynucleotide detecting cell according to the invention, a space to hold a reagent solution to be involved in chemical reactions and ECL reactions is configured between a DNA detecting cell base plate over which a working electrode to which different DNA probes are fixed is formed over a plurality of luminous areas and a transparent upper DNA detecting cell plate over which counter electrodes in a prescribed shape are formed.

The assay apparatus using the polynucleotide detecting cell according to the invention carries out a reaction to extend the DNA probe fixed to a luminous area and hybridized with a target DNA fragment, and carries out detection of the resultant extended chain by using the ECL reaction.

The assay apparatus according to the invention, as it controls at high speed the progress and stopping of the ECL reaction through high speed control of the voltage applied between the working electrode and counter electrodes, can detect at high speed the presence or absence of any extended chain formed in each luminous area. Thus it can assay a large number of probes in a short period of time with a simple apparatus configuration.

The assay apparatus according to the invention first causes a hybridizing reaction to take place between a group of DNA fragments obtained from a DNA sample with DNA probes each fixed to a luminous area, and traps a DNA fragment in each luminous area. Then, an extending reaction is carried out using adenine, thymine, guacin, cytosine and Taq DNA polymerase to each of which an ECL label is coupled, the DNA probe hybridized with an DNA fragment trapped in each luminous area is thereby extended, and dNTP (N=A, T, G, C) to which an ECL label is coupled is taken into the extended chain. A reductant is introduced into the DNA detecting cell, a voltage is applied between the working electrode and the counter electrodes, and ECL that arises on and in the vicinity of the working electrode is measured. The position of the luminous area in which ECL arises and the intensity of ECL are detected separately for each luminous area of the working electrode by combined use of an optical transmission means such as an optical fiber and a solid optical detector or of a micro-channel plate performing optical amplification and a TV camera or the like.

In the assay apparatus according to the invention, by configuring a polynucleotide detecting cell for applying a voltage to localized positions between selected luminous areas of the working electrode and the counter electrodes in an integrated manner, and measuring ECL arising in each localized position, the presence or absence of any target DNA fragment to hybridize with a DNA probe fixed to each luminous area can be quickly detected.

The assay apparatus according to the invention can use a DNA fragment group obtained by amplifying target DNA fragments by PCR employing a primer to which an ECL label is coupled, and a DNA fragment group obtained by coupling an oligomer to which an ECL label is coupled to each DNA fragment by a ligating reaction. In this case, adenine, thymine, guacin, cytosine and Taq DNA polymerase to which no ECL label is coupled is used to carry out a reaction to extend the DNA probes to be hybridized with DNA fragments.

An assay method according to a first aspect of the invention is characterized in that it has a step to trap target polynucleotides by hybridizing DNA probes fixed to luminous areas in a polynucleotide detecting cell, which is provided with a first electrode in which different DNA probes are fixed to luminous areas differing with the type of DNA probe and a second electrode opposite to the first electrode, with the target polynucleotide; a step to subject the hybridized DNA probes to an extending reaction using an ECL-labeled base to extend the DNA probes; a step to apply a voltage between the first electrode and the second electrode; and a step to detect the presence or absence of any extended chain generated by the extending reaction by detecting the presence or absence of ECL resulting from the application of the voltage.

An assay method according to a second aspect of the invention is characterized in that it has a step to trap target polynucleotides by hybridizing DNA probes fixed to luminous areas differing with the type of DNA probe in a polynucleotide detecting cell, which is provided with a first electrode and a second electrode opposite to the first electrode, with the target polynucleotides to each of which an ECL-labeled oligonucleotide is coupled; and a step to apply a voltage between the first electrode and the second electrode and thereby detect any ECL resulting from the application of the voltage.

An assay method according to a third aspect of the invention is characterized in that it has a step to trap target polynucleotides by hybridizing DNA probes fixed to luminous areas in a polynucleotide detecting cell, which is provided with a first electrode in which different DNA probes are fixed to luminous areas differing with the type of DNA probe and a second electrode opposite to the first electrode, with the ECL-labeled target polynucleotides; and a step to apply a voltage between the first electrode and the second electrode and thereby detect any ECL resulting from the application of the voltage.

Since any configuration according to the invention utilizes ECL, it can bring an optical system and an optical detector close to the DNA detecting cell in a simpler configuration than the configuration according to the prior art by which a fluorescent label is used and the fluorescent label is excited by an exciting light, and makes possible maximization of the efficiency of ECL detection.

The DNA detecting cell and the assay apparatus according to the invention, even if a very wide variety of DNA probes are used, assaying can be accomplished in only a short period of time, making possible high speed assaying. Since the ECL measuring system requires no mechanically or optically movable element, handling and adjustment can be carried out in a simple procedure.

### BRIEF DESCRIPTION OF THE DRAWINGS

FIG. 1 illustrates the configuration of a DNA detecting cell, which is the first embodiment of the present invention.

FIG. 2 illustrates a hybrid resulting from the hybridization of DNA probes fixed to a luminous area of the DNA detecting cell and part of the base sequence of target DNA fragments in the first embodiment of the invention.

FIG. 3 illustrates dATP to which a ruthenium complex for use in an extending reaction in the first embodiment of the invention is coupled.

FIG. 4 illustrates dCTP to which a ruthenium complex for use in an extending reaction in the first embodiment of the invention is coupled.

FIG. 5 illustrates dGTP to which a ruthenium complex for use in an extending reaction in the first embodiment of the invention is coupled.

FIG. 6 illustrates dTTP to which a ruthenium complex for use in an extending reaction in the first embodiment of the invention is coupled.

FIG. 7 illustrates the extending reaction of a DNA probe hybridized with a target DNA fragment in the first embodiment of the invention.

FIG. 8 shows an example of ECL detection system in the first embodiment of the invention.

FIG. 9 illustrates an example of display screen showing the detection result in the first embodiment of the invention.

FIG. 10 illustrates an example of configuration of an assay apparatus for measuring ECL in the vicinity of the working electrode of a DNA detecting cell with a TV camera in the second embodiment of the invention.

FIG. 11 illustrates the configuration of a DNA detecting cell in which a working electrode and a counter electrode are formed on the same plane in the third embodiment of the invention.

FIG. 12 illustrates the configuration of a DNA detecting cell in which the working electrode and a plurality of independent counter electrodes are formed on the same plane in the fourth embodiment of the invention.

FIG. 13 illustrates an optical system which performs detection by condensing ECL from a plurality of luminous areas in the fourth embodiment of the invention.

FIG. 14 illustrates the relationship, where ECL from luminous areas of a DNA detecting cell is to be detected with a TV camera, between the size of a luminous area as viewed on the pickup screen of the TV camera and that of a pickup element in the fifth embodiment of the invention.

FIG. 15 illustrates the configuration of a DNA detecting cell in which counter electrodes are connected by wiring of a matrix pattern and which is formed on a DNA cell base plate in the fifth embodiment of the invention.

FIG. 16 illustrates how a luminous area to induce ECL is selected by selecting gate lines and signal lines in the fifth embodiment of the invention.

FIG. 17 illustrates an example of voltage application to generate ECL repeatedly in a selected luminous area in the sixth embodiment of the invention.

FIG. 18 illustrates an oligonucleotide having phosphorothioate between 2′-deoxyoligonucleosides to be used as a DNA probe in the seventh embodiment of the invention.

FIG. 19 illustrates an assay apparatus using a DNA detecting cell in the eighth embodiment of the invention.

FIG. 20 is a plan of the DNA detecting cell for use in the eighth embodiment of the invention.

FIG. 21 illustrates an example of voltage application to generate ECL repeatedly in the selected luminous area in the eighth embodiment of the invention.

FIG. 22 illustrates a DNA probe hybridized with a target polynucleotide to which an ECL-labeled oligonucleotide is coupled in the ninth embodiment of the invention.

FIG. 23 illustrates a DNA probe hybridized with an ECL-labeled target polynucleotide in the 10th embodiment of the invention.

FIG. 24 illustrates the procedure of assaying using an assay apparatus, which is the 11th embodiment of the invention.

FIG. 25 and FIG. 26 illustrate cases in which ruthenium complexes (two types) are used as examples of the structure of ECL label and of ECL reaction usable in every embodiment of the invention.

### BEST MODES FOR CARRYING OUT THE INVENTION

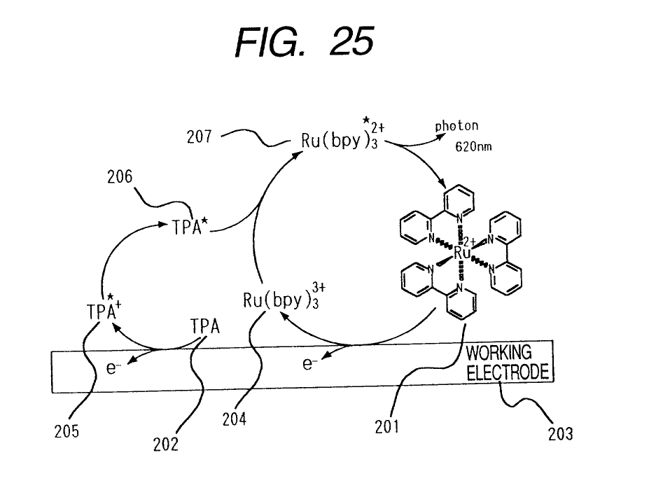

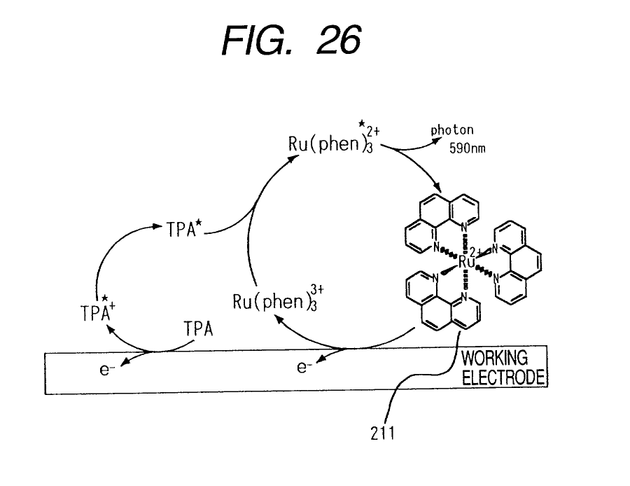

FIG. 25 and FIG. 26 illustrate cases in which ruthenium complexes (two types) are used as examples of the structure of ECL label and of ECL reaction usable in every embodiment of the present invention.

FIG. 25 illustrates an example of an ECL reaction using ruthenium(II) tris-bipyridyl (hereinafter abbreviated to Ru(bpy)₃) and tripropylamine (TPA) as the reductant (see Clinical Chemistry 37, No. 9, 1534-1539 (1991)). In a neutral solution, ruthenium(II) tris-bipyridyl (Ru(bpy)₃) is present stably in a +2-valent state (ground state) (reference numeral 201, Ru(bpy)₃⁻²). TPA 202 is present in substantial stability in a neutral solution. When a voltage is applied between a working electrode and a counter electrode so as to keep the potential of the working electrode 203 relative to the solution not below approximately +1.1 V, the ruthenium (II) tris-bipyridyl in the +2-valent state is oxidized on the surface and in the vicinity of the working electrode to enter into a +3-valent state (reference numeral 204, Ru(bpy)₃³⁻). TPA 202, too, is oxidized on the surface and in the vicinity of the working electrode to enter into a +1-valent excited state (reference numeral 205). In FIG. 25 and FIG. 26, ★ marks signify an excited state. The TPA (reference numeral 205) in the +1-valent excited state, as it is in an excited state, is turned into a TPA (reference numeral 206) in a neutral excited state by proton deprivation, and functions as a reductant on the +3-valent state (reference numeral 204, Ru(bpy)₃³⁺). The +3-valent state (reference numeral 204, Ru(bpy)₃³⁺) is reduced by the TPA in the excited state (reference numeral 206), turns into the +2-valent excited state (reference numeral 207), and returns to the +2-valent state (ground state) (reference numeral 201, Ru(bpy)₃²⁺) accompanied by ECL (whose center wavelength of luminescence distribution is about 620 nm). In the ECL reaction shown in FIG. 25, ruthenium(II) tris-bipyridyl is not consumed but is involved in the luminescence repeatedly.

FIG. 26 shows an example of an ECL reaction using ruthenium (II) tris-phenanthroline) (hereinafter abbreviated to Ru(phen)₃²⁺) (reference numeral 211) and TPA as the reductant (Clinical Chemistry 37, No. 9, 1534-1539 (1991). Electrochemiluminescence (whose center wavelength of luminescence distribution is about 590 nm) arises via a similar reaction path to that of the ECL reaction in FIG. 25.

Incidentally, besides the examples of ECL labeling shown in FIG. 25 and FIG. 26, various luminescent metal complex labels described in the Japanese Published Unexamined Patent Application No. Hei 7-173185, Published Unexamined Patent Application No. Hei 7-309836 and J. Electrochem. Soc., Vol. 132, No. 4, 842-849 (1985) can be used in the detecting apparatus according to the invention.

### First Preferred Embodiment

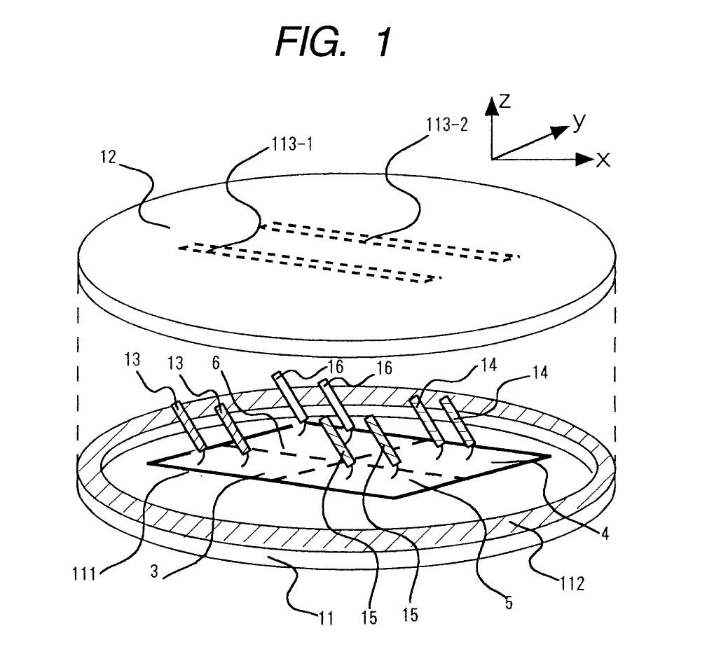

FIG. 1 illustrates the configuration of a DNA detecting cell, which is the first preferred embodiment of the present invention. The DNA detecting cell, which is the first embodiment of the invention, is configured by stacking a detection cell base plate 11 and an upper DNA detecting cell plate 12 in the z direction in FIG. 1 via a gasket 112. The space between the DNA detecting cell base plate 11 and the upper DNA detecting cell plate 12 constitutes the DNA detecting cell to hold a reagent solution to be involved in the chemical reaction and the ECL reaction used as described below. Over the upper face of the DNA detecting cell base plate 11 is formed an Au-built working electrode 111 in a prescribed shape. The upper DNA detecting cell plate 12 consists of a light-transmissive material, and on its under face are formed in parallel counter electrodes 113-1 and 113-2, each in a prescribed strip shape. The counter electrode 113-1 is opposite to luminous areas 4 and 6, and the counter electrode 113-2, opposite to luminous areas 3 and 5.

Although the DNA detecting cell base plate 11 and the upper DNA detecting cell plate 12 shown in FIG. 1 are circularly shaped, they may be otherwise shaped as desired, such as squarely, rectangularly or polygonally. Though the working electrode 111 shown in FIG. 1 is squarely shaped, it may have any other desired shape.

DNA probes (oligomers) of a plurality of types are fixed to the surface of the working electrode 111 in advance. As indicated by a chain line in FIG. 1, the face of the working electrode is divided into a plurality of luminous areas, to each of which a DNA probe of a different type is fixed, such as a DNA probe a to a luminous area a, a DNA probe b to a luminous area b, and so forth. Although each luminous area of the working electrode 111 shown in FIG. 1 is squarely shaped, it may have any other desired shape. The counter electrode 113-1 is opposite to the luminous areas 3 and 5 of working electrode 111, and the counter electrode 113-2, opposite to the luminous areas 4 and 6 of working electrode 111. Incidentally in FIG. 1, illustration of lines for voltage application to the working electrode 111 and the counter electrodes 113-1 and 113-2 is dispensed with. For efficient detection of ECL, the counter electrode 113-1 and 113-2 should desirably be transparent. Further with reference to FIG. 1, a transparent counter electrode 113 having an equal square measure to the working electrode 111 may as well be used in place of the counter electrodes 113-1 and 113-2 and arranged opposite to the working electrode 111.

In the first embodiment, DNA fragments prepared by cutting with a restriction enzyme NlaIII an 8.7 kb DNA selected from a human-derived DNA library are used, and detection is accomplished by identifying each DNA fragment with a DNA probe. The following description takes up as an example in which DNA fragments to be hybridized with first and second DNA probes are detected by using a first DNA probe 13 having a base sequence of sequence number 1 and a second DNA probe 14 having a base sequence of sequence number 2.

First DNA probe (sequence number 1):
5′TCTCACACCAGCTGTCCCAAGACCGTTTGC3′
Second DNA probe (sequence number 2):
5′AATACAGGCATCCTTCACTACATTTTCCCT3′

The first DNA probe is a probe to hybridize with a DNA fragment (first target DNA fragment) having the same base sequence as that between base 1383 and base 1927 of the aforementioned sample DNA, and the second DNA probe, a DNA fragment (second target DNA fragment) having the same base sequence as that between base 199 and base 558 of the aforementioned sample DNA. A third DNA probe 15 and a fourth DNA probe 16 are examples which do not hybridize with any position in the base sequence of the aforementioned sample DNA.

The first DNA probe 13 is fixed to the luminous area 3 of the working electrode 111, the second DNA probe 14 to the luminous area 4 of the working electrode 111, the third DNA probe 15, to the luminous area 5 of the working electrode 111, and the fourth DNA probe 16, to the luminous area 6 of the working electrode 111, in every case with a thiolic group introduced to the 5′ terminal of the DNA probe by the method described in one of the references (Biophysical Journal 71, 1079-1086 (1996)).

A sample solution containing a group of DNA fragments to be assayed is placed in the space (DNA detecting cell) between the DNA detecting cell base plate 11 and the upper DNA detecting cell plate 12 shown in FIG. 1 to hybridize the DNA probes and the DNA fragments.

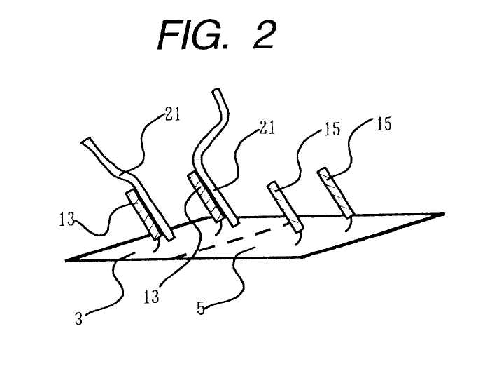

FIG. 2 illustrates a hybrid resulting from the hybridization of the first DNA probes 13 fixed to the luminous area 3 of the DNA detecting cell and part of the base sequence of target DNA fragments 21. A hybrid resulting from the hybridization of the second DNA probes 14 fixed to the luminous area 4 of the DNA detecting cell and part of the base sequence of target DNA fragments 22 not shown in FIG. 2 is formed. After the formation of the hybrids, uncombined DNA fragments are discharged out of the DNA detecting cell using a cleaning solution.

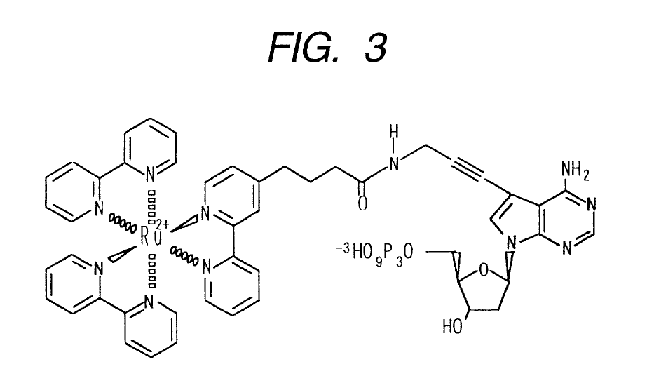

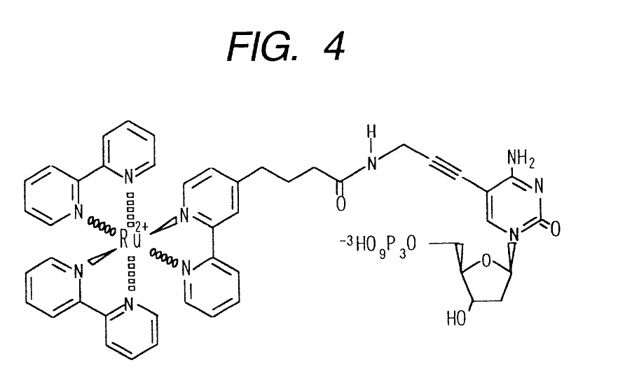

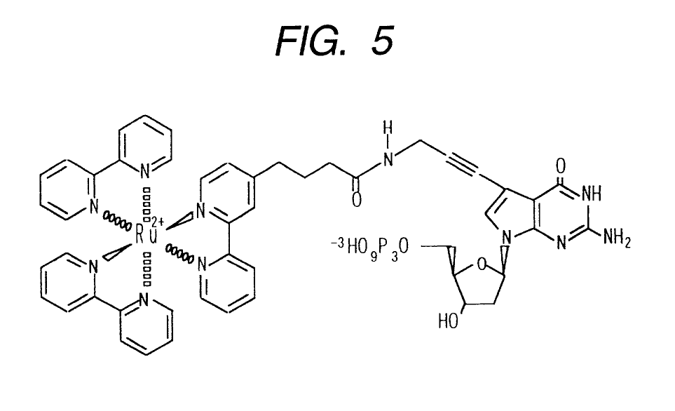

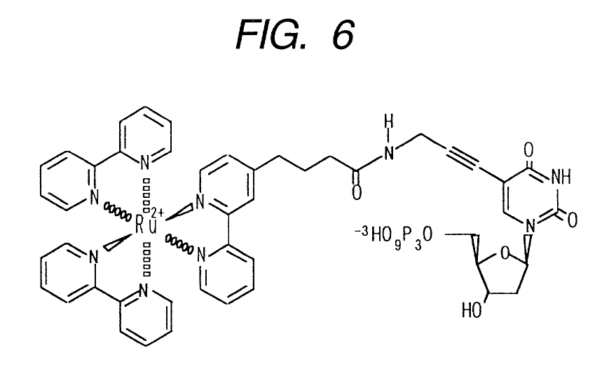

Next, a reaction to extend the first DNA probe 13 hybridized with the target DNA fragment 21 is carried out. In this extending reaction, a substrate containing at least one of dNTP (N=A, C, G, T) (abbreviated to Ru:dNTP) to which a ruthenium complex is coupled via a linker is used. As shown in FIG. 3, in Ru:dATP, a ruthenium complex is coupled to the nitrogen atom in the seventh position of adenine via a linker and a peptide bond. As shown in FIG. 4, in Ru:dCTP, a ruthenium complex is coupled to the carbon atom in the fifth position of cytosine via a linker and a peptide bond. As shown in FIG. 5, in Ru:dGTP, a ruthenium complex is coupled to the nitrogen atom in the seventh position of guanine via a linker and a peptide bond. As shown in FIG. 6, in Ru:dTTP, a ruthenium complex is coupled to the carbon atom in the fifth position of thymine via a linker and a peptide bond. The linker is —(CH₂)ₙ—, wherein n=2 to 20. A substrate mixture of any of the following compositions can be used for the extending reaction.

(1) Ru:dATP+dCTP+dGTP+dTTP

(2) dATP+Ru:dCT+dGTP+PdTTP

(3) dATP+dCTP+Ru:dGTP+dTTP

(4) dATP+dCTP+dGTP+Ru:dTTP

(5) Ru:dAT P+Ru:dCTP+dGTP+dTTP

(6) Ru:dATP+dCTP+Ru:dGTP+dTTP

(7) Ru:dATP+dCTP+dGTP+Ru:dTTP

(8) dATP+Ru:dCTP+Ru:dGTP+dTTP

(9) dATP+Ru:dCTP+dGTP+Ru:dTTP

(10) dATP+dCTP+Ru:dGTP+Ru:dTTP

(11) dATP+Ru:dCTP+Ru:dGTP+Ru:dTTP

(12) Ru:dATP+dCTP+Ru:dGTP+Ru:dTTP

(13) Ru:dATP+Ru:dCTP+dGTP+Ru:dTTP

(14) Ru:dATP+Ru:dCTP+Ru:dGTP+dTTP

(15) Ru:dATP+Ru:dTTP+Ru:dGTP+Ru:dCTP

In the first embodiment, wherein (4) of the above-listed compositions is used, 2 μL (microliters) of substrate mixture containing 2.5 mM each of dATP, dCTP, dGTPk and Ru:dTTP is added to the DNA detecting cell and, after denaturing reaction (for 10 sec) at 94° C. and annealing reaction (for 20 sec) at 66° C. once to a few times in repetition, an extending reaction is carried out at 72° C.

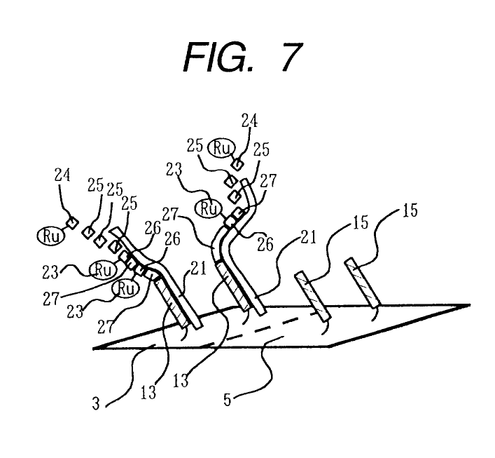

FIG. 7 illustrates the extending reaction of the first DNA probe 13 hybridized with the target DNA fragment 21 in the first embodiment of the invention. Though not shown in FIG. 7, an extending reaction similarly takes place on the second DNA probe 14 hybridized with the second target DNA fragment 22. Reference numeral 24 denotes unreacted Ru:dTTP, and 25, one of unreacted dATP, dCTP and dGTP. As a result of the extension of the first DNA probe 13 hybridized with the first target DNA fragment 21 by the extending reaction, Ru:dTTP is not accepted into the extended chain, and an extended chain consisting of an extended part 27 into whose extended chain dNTP has been accepted and an extended part 26 into whose extended chain Ru:dTTP has been accepted is formed. Therefore, the extending reaction causes at least one molecule of Ru:dTTP to be accepted into the extended chain of the first DNA probe, and at least one ruthenium complex 23 is trapped in the luminous areas 3 and 4. After the extending reaction, washing with a cleaning solution is performed to removed the unreacted substrate.

Ru:dTTP is a giant molecule compared with dNTP (N=A, C, G, T) and, though the rate of reaction of acceptance into the extended chain is lower than that of dTTP, at least its extending reaction does take place until the first molecule of Ru:dTTP is accepted into the extended chain. Since the target DNA fragments 21 and 22 are not hybridized with the third and fourth DNA probes 15 and 16, no extending reaction takes place, and Ru:dTTP is not accepted into the extended chain either. Accordingly, there is no possibility for the ruthenium complex 23 to be trapped in the luminous areas 5 and 6. Thus in the first embodiment, target DNA fragments having specific base sequences are hybridized with DNA probes fixed to the DNA detecting cell, and dNTPs to which ruthenium complexes are coupled by the extending reactions of the DNA probes are accepted into the extended chains resulting in indirect trapping of the ruthenium complexes in specific luminous areas. In the first embodiment, the quantity of the ruthenium complex 23 indirectly trapped in the luminous areas 3 and 4 is measured to detect the presence or absence of the target DNA fragment 21 in the solution containing a group of DNA fragments.

While the conventional DNA probing method using ruthenium complex-labeled DNA probes involves the problem that the labeled DNA probes are specifically adsorbed within the DNA detecting cell, Ru:dTTP used in the present invention is much less in molecular weight than the ruthenium complex-labeled DNA probes, and accordingly has the advantage that little non-specific adsorption occurs and accordingly a background attributable to non-specific adsorption can be reduced.

Next will be described how the quantity of the ruthenium complex 23 indirectly trapped in the luminous areas 3 and 4 is measured. First, the DNA detecting cell is substituted with a buffer solution containing an aminic reductant to clean the inner face of the DNA detecting cell on which the working electrode 111 and the counter electrode(s) (113-1 and 113-2 or 113) are formed. In the first embodiment, 0.30 mol/L (liter) of phosphoric acid buffer solution (pH 6.8) containing 0.18 mol/L of tripropylamine (TPA) is used as the reductant, and the temperature is kept at 28 ° C.

Next, a voltage is applied between the working electrode 111 and the counter electrode(s) (113 and 113-2 or 113) so that the working electrode 111 side be positive. The optimal voltage to be applied differs with the type of the reductant and that of the buffer solution. In the first embodiment, the voltage is applied to make the potential difference 1.35 V. The application of voltage causes an ECL reaction (the luminescence wavelength at which the intensity of the ECL caused by the ECL reaction in which the ruthenium complex used in the first embodiment is involved (the ruthenium complex part of Ru:dTTP induces the reaction of FIG. 25) is at its maximum is 620 nm) to give rise to ECL.

The intensity of ECL is proportional to the quantity of the ruthenium complex present in the vicinity of the working electrode 111. By measuring the intensity of ECL, the presence or absence of any hybrid between DNA probes and target DNA fragments can be determined.

In examples (1) through (15) of the composition of substrate mixture for use in the earlier described extending reaction, any of examples (1′) through (15′) of the composition of substrate mixture in which ddNTP (N=A, T, G, C) (abbreviated to Ru:ddNTP), wherein a ruthenium complex is coupled via a linker, is used instead of Ru:dNTP (N=A, T, G, C) may be employed.

(1′) Ru:ddATP+dCTP+dGTP+dTTP

(2′) dATP+Ru:ddCT+dGTP+PdTTP

(3′) dATP+dCTP+Ru:ddGTP+dTTP

(4′) dATP+dCTP+dGTP+Ru:ddTTP

(5′) Ru:ddATP+Ru:ddCTP+dGTP+dTTP

(6′) Ru:ddATP+dCTP+Ru:ddGTP+dTTP

(7′) Ru:ddATP+dCTP+ddGTP+Ru:dTTP

(8′) dATP+Ru:ddCTP+Ru:ddGTP+dTTP

(9′) dATP+Ru:ddCTP+dGTP+Ru:ddTTP

(10′) dATP+dCTP+Ru:ddGTP+Ru:ddTTP

(11′) dATP+Ru:ddCTP+Ru:ddGTP+Ru:ddTTP

(12′) Ru:ddATP+dCTP+Ru:ddGTP+Ru:ddTTP

(13′) Ru:ddATP+Ru:ddCTP+dGTP+Ru:ddTTP

(14′) Ru:ddATP+Ru:ddCTP+Ru:ddGTP+dTTP

(15′) Ru:ddATP+Ru:ddTTP+Ru:ddGTP+Ru:ddCTP

Where the above-listed compositions of substrate mixture (1′) through (15′) are to be used, in the extending reaction of DNA probes only one molecule of Ru:ddNTP is accepted into the extended chain. Therefore, by measuring the intensity of ECL, the quantity of hybrid of DNA probes and target DNA fragments can be quantitatively determined.

For the above-described detection of ECL is used an optical detecting system, which has a space resolving capability to detect the intensity of ECL by separating the working electrode 111 into individual luminous areas.

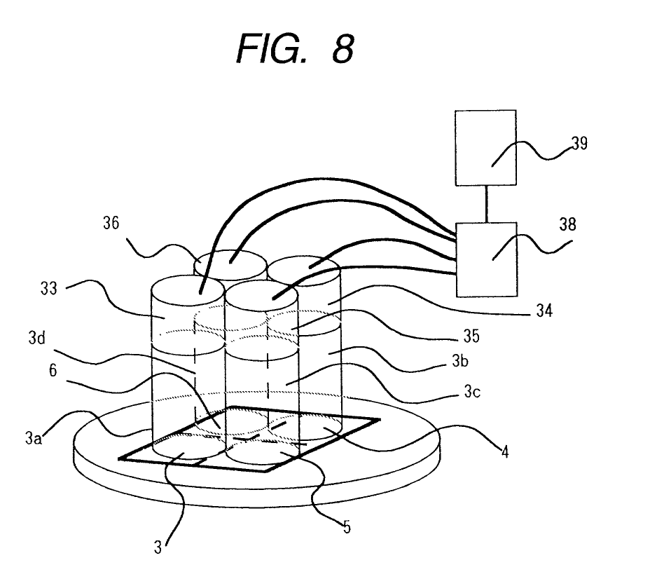

FIG. 8 shows an example of ECL detection system in the first embodiment of the invention. Here can be used an optical detection system wherein one end each of optical fibers 3a, 3b, 3c and 3d is arranged in one-to-one correspondence to one of the luminous areas 3, 4, 5 and 6, and to the other ends of the optical fibers 3a, 3b, 3c and 3d are connected high-sensitivity solid detectors 33, 34, 35 and 36, such as avalanche photodiodes (APD). The ECL that arises in the luminous areas 3, 4, 5 and 6 passes the optical fibers 3a, 3b, 3c and 3d, and is photo-electrically converted and detected by the APDs 33, 34, 35 AND 36. The outputs of the APDs are digitally converted by an A/D converter 38 and processed by a data processor 39, and the types of target DNA fragments present in the DNA fragment group can be determined from the intensity of the detected ECL in each luminous area on the basis of the above-explained principle. The determined results are displayed on the display unit of the data processor 39.

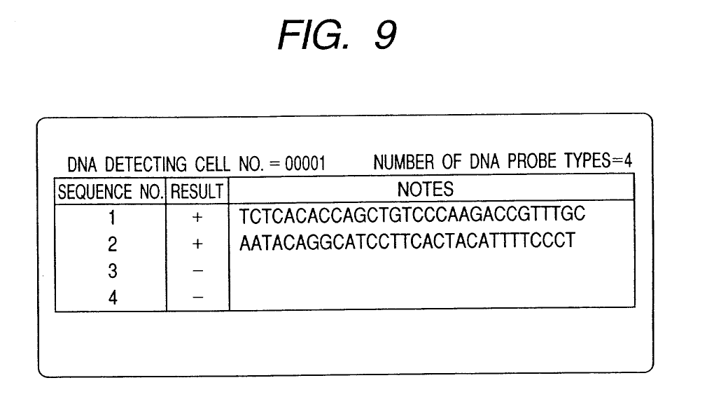

FIG. 9 illustrates an example of display screen showing the detection result on the display unit. On the screen are displayed the referenced number of the DNA detecting cell used, the number of DNA probe types fixed to the DNA detecting cell (the number of luminous areas), the sequence No. of the DNA probe fixed to each luminous area, and the result of assaying of the reaction of the DNA probe with any DNA in the sample {+(positive: DNA probe) or -(negative: there is in the sample no DNA forming a hybrid with the DNA probe) }. If the result is positive, the base sequence of the DNA probe is displayed. The example in FIG. 9 shows that DNAs having base sequences hybridizing with DNA probes of 1 and 2 in sequence number have been detected.

Although in the description of the first embodiment, for the sake of brevity, an example in which a DNA detecting cell having four luminous areas was cited, the number of luminous areas in a DNA detecting cell in an actual assay apparatus can be, as will be described with reference to the second embodiment for instance, 100×100=10000. The number of luminous areas is selected to suit the purpose of assaying.

Incidentally, instead of a ruthenium complex label, some other label used in ECL reactions, such as an osmium complex, can be used as well. For instance in the first embodiment, Os:dTTP and Os:ddTTP may be used in place of Ru:dTTP and Ru:ddTTP.

### Second Preferred Embodiment

Where a DNA detecting cell is fabricated by large-scale integration to enable more DNA probes to be handled at a time (i.e., where the number of DNA luminous areas in the DNA detecting cell is increased) , a method of detecting ECL with a 2D imaging device, such as a high sensitivity camera, is effective, whereby the distribution of ECL in the vicinity of the working electrode of the DNA detecting cell can be taken in as a 2D image and a large quantity of data can be handled at a time by image processing.

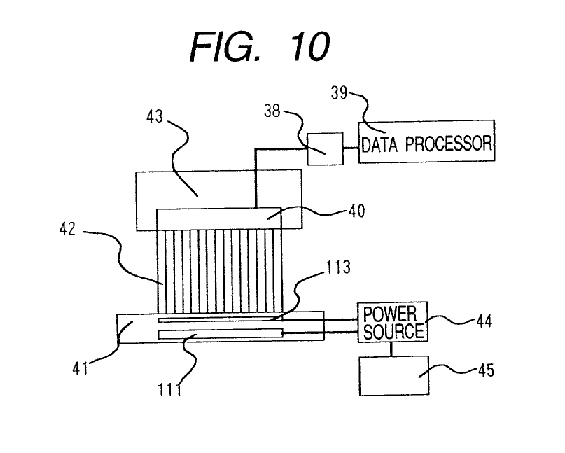

FIG. 10 illustrates an assay apparatus for measuring ECL in the vicinity of the working electrode 111 of the DNA detecting cell 41 with an optical system 42 and a TV camera 43 having a plurality of pickup elements 40. A transparent counter electrode 113 arranged opposite and having an equal square measure to the working electrode 111 , a voltage applied by a power source 44 between the counter electrode 113 and the working electrode 111, and the duration of voltage application (0.4 sec in the second embodiment) are controlled by a power source controller 45. The optical system 42 may use an ordinary optical lens, but it would be more effective to increase the optical detection sensitivity by using an image intensifier (I.I.) or a micro-channel plate (MCP). Further, where higher resolution performance is necessitated by the larger scale integration of the DNA detecting cell, an optical system in which an optical fiber flux is directly connected to the image pickup screen of the TV camera 43 should be used.

In the DNA detecting cell of the second embodiment, the 20 mm×20 mm face of the working electrode is divided into 100×100=10000 luminous areas each having a square measure of 200 μm×200 μm and arranged in the x and y directions, and a different DNA probe is fixed to each luminous area. Supposing that one molecule of DNA probe is fixed in an area of approximately 50 nm×50 nm, about 16 million molecules (0.027 fmol) of DNA probe are fixed to each luminous area having a square measure of 200 μm×200 μm.

Here is taken up as an example a case in which, as a result of the extending reaction of a DNA probe hybridized with a target DNA fragment, one molecule alone of dNTP or ddNTP to which an ECL label per molecule of DNA probe is coupled has been taken into the extended chain. Where the distance between the working electrode and the upper cell plate is 200 μm, an ECL label of about 3.3 nmol/L in concentration is indirectly trapped in one luminous area (200 μm square) (0.027×10⁻¹⁵ mol/(200×10⁻⁴ cm)³≈3.3 nmol/L).

According to one of the References (Clinical Chemistry 37, No. 9, 1534-1539 (1991)), in ECL tris-bipyridyl using ruthenium tris-bipyridyl complex and TPA, the detectable limit is 200 fmol/L, and accordingly in the second embodiment there should be about 16,500 times more ECL labels (3.3×10⁻⁹÷(200×10⁻¹⁵)≈16500) per luminous area than the detectable limit (200 fmol/L).

Where the square measure of the working electrode is 4 mm×5 mm, the number of photons actually detected in an experiment by measuring ECL in a solution whose ruthenium tris-bipyridyl complex concentration was 10 nmol/L for 0.4 sec was about 4000 (CV=0.5%) per mm² of the working electrode, though the apparatus used in the experiment used no optical system for condensing ECL, and the efficiency of ECL detection, represented by the product of the multiplication of the ratio between the solid angle of the area of each luminous area with respect to that of the light receiving area of the PMT, which is the optical detector, and 2π (str) by the quantum efficiency of the PMT (5% here), is about 0.6%.

Therefore, by using a DNA detecting cell wherein the 20 mm×20 mm area of the working electrode in this second embodiment is divided into 100×100=10000 luminous areas each having a square measure of 200 μm×200 μm, the F value of the lens to condense ECL being 0.65 and the quantum efficiency of the cooling CCD camera being 10%, the efficiency of ECL detection will be about 0.7%, and the number of photons detected per luminous area in 0.4 sec (ECL quantity) will be about 50 (4000/{(1000 μm)²×(10 nmol/L)}×{({200 μm)²×(3.3 nmol/L)}≈52.8). Supposing that the ECL quantity is proportional to the concentration of the ECL label (complex) and the S/N ratio is proportional to the square root of the ECL quantity (S), the S/N ratio in the measurement with the second embodiment will be about 7. Where the configuration of the DNA detecting cell used in the experiment is such that the 10 mm×10 mm area of the working electrode is divided into 2500 luminous areas each having a square measure of 200 μm×200 μm and the aforementioned lens and cooling CCD camera are used, the light condensing efficiency of ECL is approximately doubled, the number of photons detected per luminous area in 0.4 sec (ECL quantity) will increase to about 100, and the S/N ratio is improved to about 10. Whereas the foregoing description referred to the S/N ratio in a case where one molecule of dNTP or ddNTP to which an ECL label is coupled is taken into the extended chain of one molecule DNA probe, where n molecules of dNTP to which an ECL label is coupled is taken into the extended chain of one molecule DNA probe, the S/N ratio will be √n times the aforementioned value.

An MCP is used as the optical system 42 and one million pixel cooling CCD camera is used as the 2D detector to photograph the distribution of luminescence on the working electrode. The signal charges accumulated in the elements 40 are converted into amperages or voltages, and digitized by the A/D converter 38 to obtain a 2D digital image. The obtained 2D digital image is binarized by the data processor 39, and differentiated into active luminous areas and inactive luminous areas. The types of DNA fragments present in the sample can be identified by comparing probe information on each luminous area indicating what kind of DNA probe is fixed in a luminous area of what position and luminescence information (the presence or absence of luminescence and the intensity of luminescence) are compared.

As hitherto described, by using a one million pixel cooling CCD camera, the presence or absence of hybrids between 10000 different types of DNA probes and target DNA fragments in the sample can be detected in a measuring time of 0.4 sec. From the intensity of ECL detected in each luminous area, the types and quantities of target DNA fragments present in the DNA fragment group can be determined in accordance with the principle explained with reference to the first embodiment.

### Third Preferred Embodiment

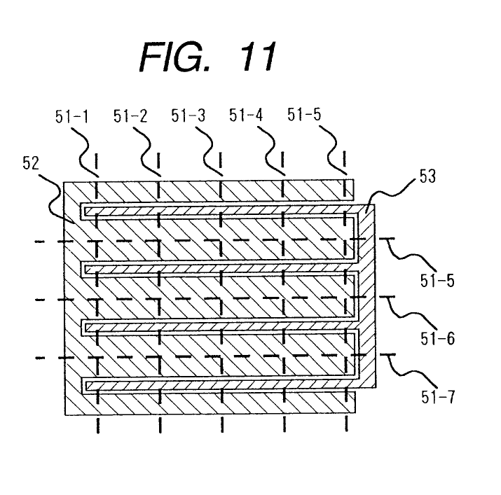

FIG. 11 illustrates the configuration of a DNA detecting cell in which a comb-shaped working electrode and a comb-shaped counter electrode are formed on the same plane. FIG. 11 shows the working electrode as viewed from the optical detector side. In this third embodiment of the invention, the working electrode 52 and the counter electrode 53 are formed on the same plane to enhance the efficiency of ECL detection. The comb-shaped working electrode 52 is partitioned by broken lines 51-1 through 51-7 into a plurality of luminous areas. The total number of the luminous areas (each measuring 200 μm×200 μm) , as in the second embodiment, is 10000. The comb-shaped working electrode 52 and the comb-shaped counter electrode 53 are so formed on the surface of the DNA cell base plate so that the teeth of the comb-shaped working electrode 52 and those of the comb-shaped counter electrode 53 alternately oppose each other in one direction.

Thus the luminous areas of the working electrode 52 are arranged in one direction in mesh with the counter electrode 53 (5 μm wide) with 5 μm gaps between them. As this arrangement results in substantially uniform voltage application to the luminous areas, there is no variation in the total of ECL intensities generating from the luminous areas. In this third embodiment, as there is no need to provide a counter electrode on the upper cell plate matching the DNA cell base plate and therefore the propagation of ECL is not intercepted, it is made possible to enhance the efficiency of ECL detection.

Incidentally, in the third embodiment, voltage is applied between the working electrode 52 and the counter electrode 53 under the same conditions as in the second embodiment. The time taken to measure ECL is 0.4 sec.

### Fourth Preferred Embodiment

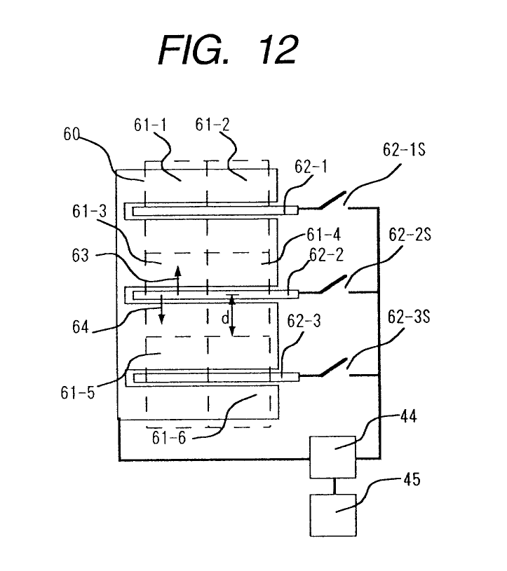

FIG. 12 illustrates the configuration of a DNA detecting cell in which the working electrode and a plurality of independent counter electrodes are formed on the same plane. With reference to this fourth embodiment of the invention, a configuration of a DNA detecting cell in which luminous areas are integrated beyond the resolution performance of the optical detector will be described. The electrode arrangement in the DNA detecting cell in the fourth embodiment, though resembling that in the third embodiment, the total number of luminous areas (each measuring 200 μm×200 μm) partitioned by broken lines is 10000 as in the second embodiment (of which only six luminous areas are shown in FIG. 12). In this configuration, teeth of a working electrode 60 are arranged to hold between them counter electrodes 62-1, 62-2 and 62-3 (5 μm wide) with 5 μm gaps between them in one direction, and this embodiment differs from the third in that voltage can be separately applied to the counter electrodes 62-1, 62-2 and 62-3. The working electrode 60 is kept at the grounding potential all the time, and voltage can be separately applied to the counter electrodes 62-1, 62-2 and 62-3 by the power source controller 45 and the power source 44 using switches 62-1S, 62-2S and 62-3S.

The working electrode 60 is partitioned by broken lines into luminous areas 61-1 through 61-6. When the potential of every counter electrode is 0 V, the potential of the solution within the DNA detecting cell is uniformly 0 V. When the potential of the counter electrode 62-2 is varied from 0 V to −1.4 V, the uniformity of the solution in potential is lost, and the solution potential in the vicinity of the counter electrode 61 is reduced from 0 V to a negative voltage. This negative potential of the solution spreads radially over time from the counter electrode 62-2. Whereas the potential distribution of the electric double layer formed in the vicinity of the working electrode varies, as if to reflect the potential between the solution and the working electrode, when the solution potential on the working electrode surface becomes negative, the variation in the potential distribution of the electric double layer propagates at a velocity of V_{T} in the directions of arrows 63 and 64 with the spread of the negative solution potential. The transmission speed V_{T}, which differs with the ion concentration of the solution, the temperature of the solution and the potential difference between the solution and the working electrode among other factors was about 15 mm/sec where, for instance, phosphoric acid buffer solution (pH6.8) of 0.30 mol/L containing tripropylamine (TPA) of 0.18 mol/L was used as the reductant. Where the potential distribution of the electric double layer is formed so as to keep the potential difference between the solution and the working electrode not less than −1.1 V, an ECL reaction arises between a ruthenium complex and TPA. The luminescent region expands from the counter electrode 62-2 at a velocity of about 15 mm/sec in the directions of the arrows 63 and 64.

When a period of time T=d/V_{T}, where d is the distance between the center of the counter electrode and the boundary of a luminous area, has elapsed after the counter electrode 62 is set to a negative potential, the variation in the potential distribution of the electric double layer formed in the vicinity of the working electrode surface due to a variation in the potential of the counter electrode 62-2 reaches the boundary of the luminous area. As a result, in the luminous area, out of the luminous area 61-3 and the luminous area 61-4, where an ECL label has been trapped, an electrochemical reaction occurs, giving rise to ECL. As the counter electrode 62-2 is returned to the grounding potential when the period of time T=d/V_{T} has elapsed after the counter electrode 62 is set to a negative potential, the propagation of the potential difference between the solution and the electrode is discontinued, and no other luminous area will generate ECL under the impact from the counter electrode 62-2. Similarly, if any other counter electrode is selected and a potential is provided for a period of T, an ECL reaction can be excited only in a luminous area containing the selected counter electrode. The fourth embodiment uses an optical detection system in which three luminous areas constitute one unit of optical detection.

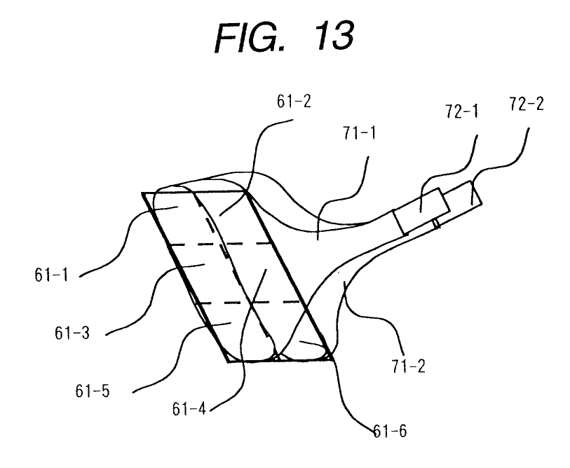

FIG. 13 illustrates an optical system which performs detection by condensing ECL from a plurality of luminous areas and detecting the condensed ECL. The total number of the luminous area is 10000 as in the second embodiment, only six of which are shown in FIG. 13. In the configuration illustrated in FIG. 13, ECL from luminous areas 61-1, 61-3 and 61-5 is condensed by an optical fiber 71-1 into an APD 72-1, and ECL from luminous areas 61-2, 61-4 and 61-6 is condensed by an optical fiber 71-2 into an APD 72-2 (for ECL detection from 10000 luminous areas, 100 APDs are required). It is possible to measure ECL from six luminous areas with two APDs by selecting one counter electrode at a time with a switch, providing it with a negative potential and reading optical detection signals from two APDs in synchronism with the selection of a counter electrode. While the light receiving faces of the optical fibers 71-1 and 72-2 are greater in square measure than each luminous area, this fourth embodiment can successively measure ECL from a plurality of luminous areas with a single optical detection system by selecting a counter electrode at a time, and accordingly has the advantage of being able to evaluate a DNA detecting cell having a greater number of probe types than the total number of optical detection systems.

Incidentally, with this fourth embodiment, after successively selecting and switching on a counter electrode, a voltage (e.g. −1.4 V) is applied between the working electrode 60 and one of the counter electrodes 62-1 through 62-3 and so forth. Where the size of each luminous area is 200 μm×200 μm and the transmission speed V_{T} is about 15 mm/sec as in the second embodiment, the duration of the aforementioned voltage application is (0.2 mm/2)/15=0.0067 sec=6.7 msec or less, and the length of time required for detecting ECL from 10000 luminous areas is 6.7 msec×100=0.67 sec.

### Fifth Preferred Embodiment

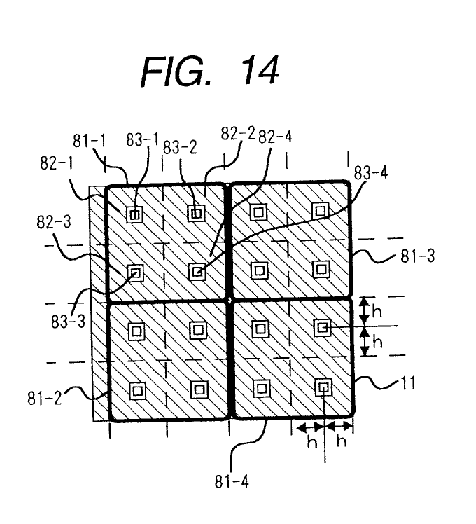

FIG. 14 illustrates the relationship, where ECL from luminous areas of an integrated DNA detecting cell is to be condensed and detected with a TV camera, between the size of a luminous area as viewed on the pickup screen of the TV camera and that of a pickup element. Use of a TV camera as the optical detector is effective where a DNA detecting cell having many partitions for using many types of DNA probes at a time. A working electrode 111 (formed on the detection cell base plate) shadowed in FIG. 14 is divided into a plurality of luminous areas (measuring 200 μm×200 μm in external shape) by broken lines in the x and y directions, and in the central part of each luminous area is arranged a counter electrode (externally shaped in a square each side of which measures 5 μm to 10 μm) with a few μm's spacing, surrounded by and opposite to the working electrode 111 (shaded). Thus the counter electrodes are formed separately from the working electrode 111 (shaded) on the DNA cell base plate 12. The distance between the center of each counter electrode and the boundary of each luminous area is represented by h (=100 μm). The total number of luminous areas is 10000 as in the second embodiment, the external dimensions of the working electrode are 20 mm×20 mm, and in FIG. 14 only 16 luminous areas are shown. In the following description will be considered a case in which ECL from a total of four luminous areas, 82-1 through 82-4, comes incident on the detectable area (one factor defining resolution performance) of a first detection (pickup) element 81-1 in an image observed on the pickup screen of an optical detector, for instance a TV camera.

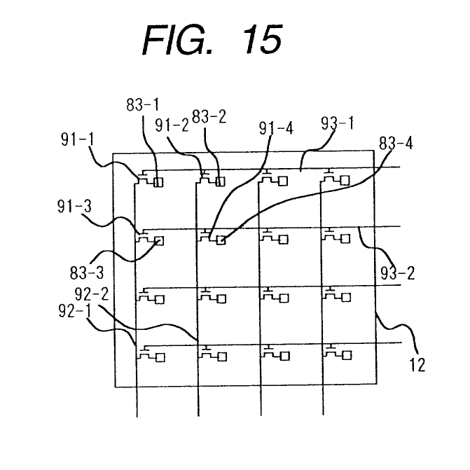

FIG. 15 illustrates the configuration of a DNA detecting cell in which counter electrodes are connected by wiring in a matrix pattern and which is formed on a DNA cell base plate. On the surface of the DNA cell base plate (electrically insulating material) are formed wiring and gates shown in FIG. 15, then the working electrode and counter electrodes shown in FIG. 14 are formed via an insulating layer, and the counter electrodes and the gates are electrically connected. Although the total number of the counter electrodes is 10000, only 16 of them are shown in FIG. 15 as in FIG. 14. As illustrated in FIG. 15, the matrix wiring consists of TFT gates 91-1 through 91-4 respectively matching the counter electrodes 83-1 through 83-4, a signal line 92-1 connected to the gates 91-1 and 91-3, a signal line 92-2 connected to the gates 91-2 and 91-4, a gate line 93-1 for controlling the turning on and off the gates 91-1 and 91-2, and a gate line 93-2 for controlling the turning on and off the gates 91-3 and 91-4.

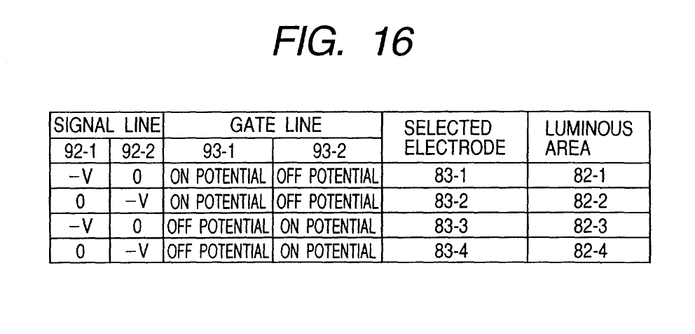

FIG. 16 illustrates how a counter electrode, i.e. a luminous area to induce ECL, is determined by selecting gate lines (93-1 and 93-2) and signal lines (92-1 and 92-2). In the DNA detecting cell shown in FIG. 14 and FIG. 15, when other gate lines than the gate line 93-1 are set to an OFF potential (e.g. a 0 potential) and the gate line 93-1 is held at an ON potential (e.g. 10 V or above) for a period of time T=h/V_{T} in a state wherein other signal lines than the signal line 92-1 are set to a 0 potential and a negative potential is applied to the signal line 92-1, the gate 91-1 connected to the gate line 93-1 is turned ON, and the signal line 92-1 and the counter electrode 83-1 become electrically continuous. As a result, because an electric double layer can be so formed that the counter electrode 83-1 take on a negative potential only as long as the period of time T and the potential difference between the solution and the electrode be set to −1.4 V only in the luminous area 82-1, an ECL reaction takes place if an ECL label is trapped in the luminous area 82-1. Incidentally if a negative potential is applied to every signal line connected to the counter electrodes in the upper left luminous areas (in FIG. 14, for the sake of brevity, a reference numeral is assigned to only 82-1) in the detectable areas 81-1 through 81-4 of the first through fourth pickup elements and an ON potential is applied to every gate line connected to the gates of the upper left luminous areas, ECL can be selectively induced only in the upper left luminous areas in the detectable areas 81-1 through 81-4.

Hereafter, by selecting gate lines and signal lines as shown in FIG. 16, it is possible to select a counter electrode, i.e. a face on which ECL is to be induced (a luminous area) , and successively detect on an area-by-area basis ECL from luminous areas belonging to the detectable areas 81-1 through 81-4. For instance, ECL from the four luminous areas 82-1 through 82-4 belonging to the detectable area 81-1 of the first pickup element can be successively detected, separated on an area-by-area basis. As a result, in the fifth embodiment, in spite of the greater detectable area per detecting element of the optical detector than each luminous area of the DNA detecting cell, the number of luminous areas detectable with a single detecting element is four.

Incidentally, in the fifth embodiment, ECL from four different luminous areas at a time is detected with one detecting (pickup) element of the TV camera, switched at intervals. Thus, ECL from 10000 luminous areas is detected in four rounds. A voltage (e.g., −1.4 V) is applied between the working electrode 111 and the counter electrodes 83-1 through 83-2 and so forth. When the size of each luminous area is 200 μm×200 μm as in the second embodiment and the transmission speed V_{T} is about 15 mm/sec as in the fourth embodiment, the duration T of the above-mentioned voltage application is no longer than 6.7 msec, and the length of time taken to detect ECL from 10000 luminous areas is 6.7 msec×4=26.8 msec.

With reference to the fifth embodiment described above, an example in which a cooling CCD camera is used as the TV camera will be described below, where a one-inch cooling CCD camera consisting of one million elements (the size of each CCD element on the light receiving face of the CCD camera is 18 μm). The reduction rate of the optical system arranged between the CCD camera and the DNA detecting cell is supposed to be about 1/11. The resolution performance achieved where a TV camera, such as a CCD camera, is used is, to put it in simple terms from a practical point of view, is about 2L, where L is the size of the optical detecting element on the light receiving face. Where L=18 μm, the configuration will be such that four CCD elements detect ECL from four luminous areas of the DNA detecting cell. Here the configuration is supposed to be one for detection of ECL separately from four luminous areas by a method similar to the above-described. The four CCD elements form a first optical detection opening (it is used in the sense that the four elements constitute one effective element; it corresponds to 81-1 shown in FIG. 14), and similarly each of other second through fourth optical detection openings 81-2 through 81-4 is formed of four elements. Since each optical detection opening is formed of four CCD elements, where a one-inch CCD camera consisting of one million elements is to be used, each optical detection opening consists of a total of 250,000 elements. The size of the working electrode of the DNA detecting cell earlier described is enlarged from the 20 mm to 200 mm, one million luminous areas are formed, and a different probe is fixed to each luminous area. The length of time taken to detect ECL from the DNA detecting cell having one million luminous areas using a one-inch CCD camera consisting of one million elements is 26.8 msec as in the above-described case.

This configuration of the fifth embodiment makes possible measurement in as short a period as 26.8 msec of ECL from each luminous area independently of others, irrespective of the number of luminous areas constituting the DNA detecting cell. Each optical detection opening is formed of a plurality of CCD elements, and ECL from a plurality of luminous areas covered by each optical detection opening can be detected in high resolution performance for each luminous area independently of others.

### Sixth Preferred Embodiment

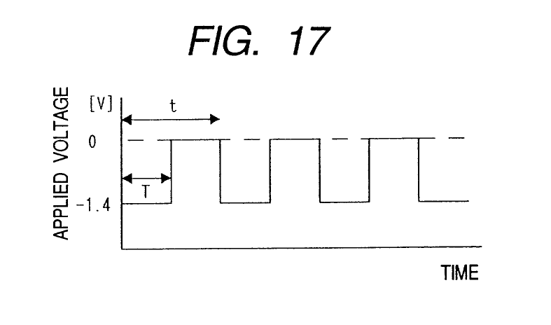

FIG. 17 illustrates an example of voltage application to generate ECL repeatedly in one selected luminous area. This sixth embodiment of the invention is similar to the fourth and fifth embodiments except that, after a prescribed period of relaxation, a negative potential is repeatedly applied to the counter electrode to induce an ECL reaction again. If the duration T of applying a negative potential to the counter electrode and one cycle of the period t cannot achieve a sufficient ECL intensity and the detection sensitivity of the detecting elements of the optical detector cannot be reached, a negative potential is repeatedly applied to the counter electrode after a prescribed period of relaxation to re-induce an ECL reaction and, as shown in FIG. 17 for instance, the voltage is applied repeatedly a plurality of times to one counter electrode in the period t to store light in the detecting elements of the optical detector and thereby detect ECL. Control of the voltage application is accomplished by the power source controller 45 through its control of the power source 44.

If for instance in the fifth embodiment h=100 μm and V_{T}=15 mm/sec, the duration T of voltage application will be T=6.7 msec. Where the period is extended to t=2T=13.4 msec, the applied voltage can be controlled with a 75 Hz rectangular wave (the aforementioned period of relaxation will then be 6.7 msec) . As a result, an integrated intensity of ECL by the repetition of the ECL reaction is obtained, and the problem of insufficient ECL intensity in one cycle alone (low detection sensitivity and low S/N ratio) can be solved. Where the voltage application shown in FIG. 17 is repeated n cycles using a DNA detecting cell having 10000 luminous areas, the length of measurement time required for ECL detection will be 0.67×(2 n) sec in the fourth embodiment or 26.8×(2 n) msec in the fifth embodiment, providing n times as great an ECL intensity as that obtained in one cycle alone and √n times as high an S/N ratio. If for instance n=60, measurement time will be 80.4 sec (in the fourth embodiment) or 3.2 sec (in the fifth embodiment).

It is the same as in the first and second embodiments that, in the third through sixth embodiments, signals detected by the optical detector are converted into a current or a voltage, digitally converted by the A/D converter 38 and processed by the data processor 39.

### Seventh Preferred Embodiment

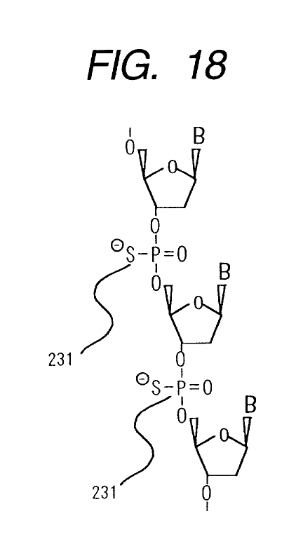

In the first through sixth embodiments, the DNA probes 13, 14, 15 and 16 fixed to the DNA detecting cell are oligonucleotides each having a phosphoric acid diester bond between 2′-deoxyoligonucleosides. In the ninth embodiment of the invention, as shown in FIG. 18, oligonucleotides each having a phosphorothioate bond (reference numeral 231) between 2′- deoxyoligonucleosides are used as the DNA probes 13, 14, 15 and 16 and fixed to the DNA detecting cell in the first through sixth embodiments. B shown in FIG. 18 denotes a nucleic acid base (any of A, T, G and C). DNA probes having a phosphorothioate bond is not decomposed by S1 nuclease.

### Eighth Preferred Embodiment

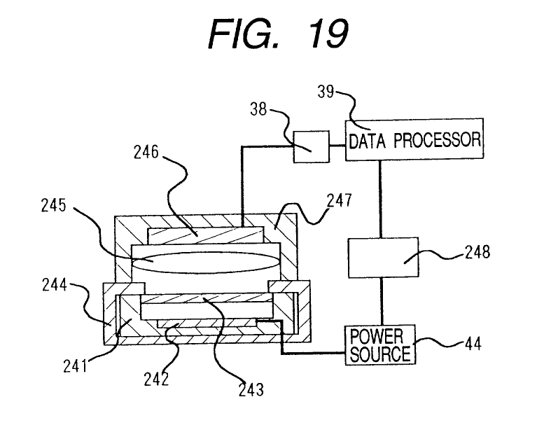

FIG. 19 illustrates an assay apparatus using a DNA detecting cell. The DNA detecting cell has a base plate 241 with a concave part and a transparent upper plate 243, and on the bottom face 242 of the concave part of the base plate 241 are arranged the working electrode and the counter electrode on the same plane as described wish reference to the third embodiment (FIG. 11). The DNA detecting cell is inserted into an optically opaque cell holder 244 and fixed there. Opposite to the upper plate 243 are arranged an image formation lens 245 and, in the image formation position of the image formation lens 245, a cooling CCD camera 246. A camera head 247 fixing the CCD camera 246 is optically opaque and, connected to the cell holder 244, intercept external light from coming incident on the DNA detecting cell and the optical system. To the working electrode and the counter electrode of the DNA detecting cell is connected the power source 44, and voltage application by the power source 44 and reading of signals accumulated in the CCD camera 246 are controlled by a controller 248. The signals that are read out are digitally converted by the A/D converter 38, processed by the data processor 39 and stored into a memory as a 2D digital image. Incidentally, in the configuration of FIG. 19, the counter electrode may as well use the configuration illustrated in FIG. 1 (first embodiment), FIG. 10 (second embodiment), FIG. 12 (fourth embodiment) or FIG. 14 (fifth embodiment).

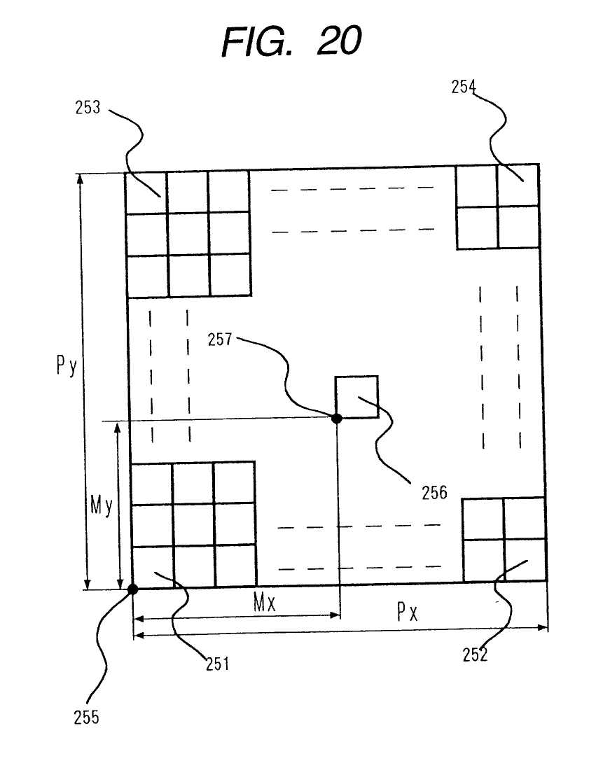

FIG. 20 is a plan of the DNA detecting cell shown in FIG. 19 as viewed from the image formation lens 245 side. The DNA detecting cell in this eighth embodiment has the same configuration as the DNA detecting cell of the second embodiment, wherein the 20 mm ×20 mm surface of the working electrode is divided into 100×100=10000 luminous areas each measuring 200 μm×200 μm, and a different DNA probe is fixed to each luminous area.

Incidentally, the eighth embodiment having the configuration described above takes 0.4 sec to accomplish ECL measurement as the second embodiment does.

As described with reference to the second embodiment, where ECL is to be obtained as a 2D digital image, or as described with reference to the fourth, fifth and sixth embodiments, where the DNA detecting cell is integrated beyond the resolution performance of the optical detector, the sequence of the luminous areas of the DNA detecting cell and the sequence of the detecting elements of the optical detector should be matched precisely or the positions of the luminous areas activated after the measurement of ECL should be determined accurately.

To facilitate the positional matching of the sequence of the luminous areas of the DNA detecting cell and the sequence of the detecting elements of the optical detector, a plurality of markers are provided on the DNA detecting cell, and the positional relationship between the DNA detecting cell and the optical detector is adjusted by utilizing the positions of the markers. Or else, the positions of the optical markers are measured at the same time as ECL measurement, and the positional relationship between the DNA detecting cell and the optical detector is detected at the time of data processing to accurately determine the positions of activated luminous areas. Either luminescence sources of minute square measures, such as light emitting diodes, are arranged on the DNA detecting cell to be used as markers, or pinholes are bored through the DNA detecting cell and light beams having passed the pinholes are used as markers. Alternatively, luminous areas in specific positions of the DNA detecting cell can be used as markers.

The four luminous areas 251, 252, 253 and 254 shown in FIG. 20, used as markers of the DNA detecting cell, are specially prepared luminous areas. Where the quantity of DNA probes to be fixed to luminous areas is 0.027 fmol as in the second embodiment, a ruthenium complex of a known concentration is fixed in quantities of 0.027 fmol to the luminous area 251, of 0.0203 fmol to the luminous area 252, of 0.0135 fmol to the luminous area 253 and of 0.0068 fmol to the luminous area 254. ECL from the luminous area to which the ruthenium complex of the known concentration is fixed can be utilized as the scale of luminescence intensity.

The ruthenium complex of the known concentration can be fixed either at the time of preparing the DNA detecting cell or by the method described below. For instance, probes having different base sequences from all other DNA probes (to be referred to as marker probes) are fixed to the luminous areas 251, 252, 253 and 254 in the aforementioned known concentration in advance and, when target DNA fragments are to be hybridized with the DNA probes of luminous areas, a marker DNA which is hybridized only with marker probes is added to hybridize the marker probes with the marker DNA, and the marker probes are subjected to an extending reaction simultaneously with the extending reaction of DNA probes described with reference to the first embodiment. This method makes it possible to confirm that the hybridization and the extending reaction have been successfully accomplished by measuring luminescence attributable to Ru:dNTP or Ru:ddNTP taken into the extended chains of the marker probes.

The positions of the activated luminous areas in the DNA detecting cell are located by the following method. ECL can be detected all the time from the four luminous areas 251, 252, 253 and 254 shown in FIG. 20. A method to analyze a detected 2D luminescent image will be described below. A 2D luminescent image containing luminescence from the luminous areas 251, 252, 253 and 254 is represented by x and y coordinates having reference numeral 255 as the origin, and the number of pixels Px in the x direction and the number of pixels Py in the y direction constituting the luminescent image are determined. Since the DNA detecting cell consists of 100×100 luminous areas, the numbers of pixels per luminous area are Px/100=Qx and Py/100=Qy. The coordinates (Mx, My) of an angular point 257 close to the origin of the active luminous area 256 are determined (Mx and My are numbers of pixels), and I=[Mx/Qx+1] and J=[My/Qy+1] are calculated ([] means that the first and second decimals of the value in the brackets are rounded, and the resultant I and J are integers). As a result, it is found that the activated luminous area 256 is the luminous area of the Jth column and the Ith row of the DNA detecting cell.

While ECL is measured for 0.4 sec by the second embodiment, the eighth embodiment repeatedly measures ECL from the same luminous area.

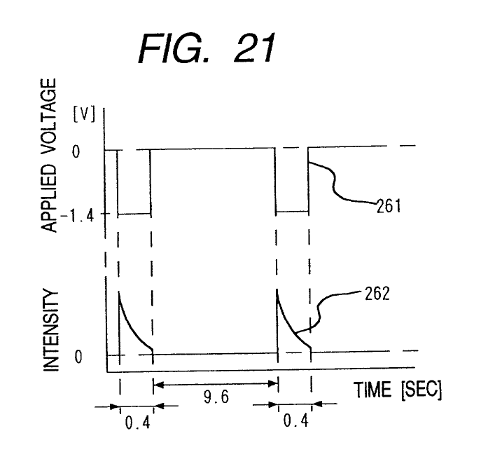

FIG. 21 illustrates an example of voltage application to generate ECL repeatedly. In FIG. 21, the horizontal axis represents time, reference numeral 261 denotes the voltage applied between the working electrode and the counter electrode of the DNA detecting cell, and reference numeral 262, the intensity from the ECL label. Whereas ECL is generated simultaneously with the application of the voltage for 0.4 sec, the reductant that is used (TPA in the eighth embodiment) is quickly consumed on the surface and in the vicinity of the working electrode, and the intensity rapidly decreases. Therefore, no increase in intensity can be expected even if the voltage is applied for a longer duration. However, if a 9.6 sec period of relaxation is set following a stop of voltage application, the reductant in the solution is supplied to the surface and the vicinity of the working electrode by diffusion. When the voltage is applied for 0.4 sec again, ECL will be generated. By alternately repeating the voltage application for 0.4 sec and the stop of voltage application for 9.6 sec (in a period of 10 sec), the total quantity of ECL can be increased, which is effective for enhancing the detection sensitivity. Incidentally, the voltage application method illustrated in FIG. 21 is not confined to the second embodiment, but can be applied to any of the first through seventh embodiments with similar effects.

### Ninth Preferred Embodiment

While Ru:dNTP or Ru:ddNTP is used to carry out the extending reaction of DNA probes hybridized with target DNA fragments in the first embodiment, the seventh embodiment uses a method involving no execution of the extending reaction. ECL-labeled oligonucleotide 28 is coupled to the target polynucleotide (target DNA fragments) 21 in advance.

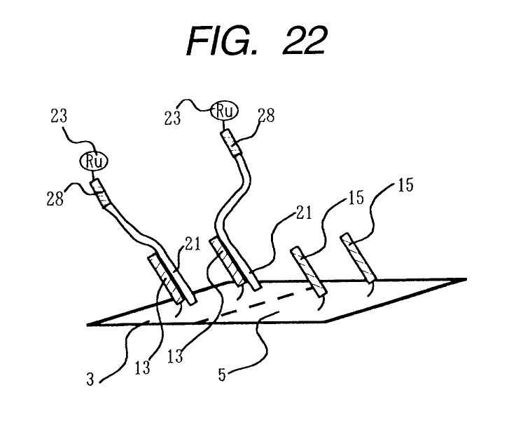

FIG. 22 illustrates a DNA probe hybridized with a target polynucleotide to which an ECL-labeled oligonucleotide is coupled. ECL is detected by the method described with reference to the first through sixth embodiments.

### 10th Preferred Embodiment

While the ninth embodiment used an ECL-labeled oligonucleotide, the ECL label may as well be coupled to the 5′ terminal side of the target polynucleotide (target DNA fragments) 21.

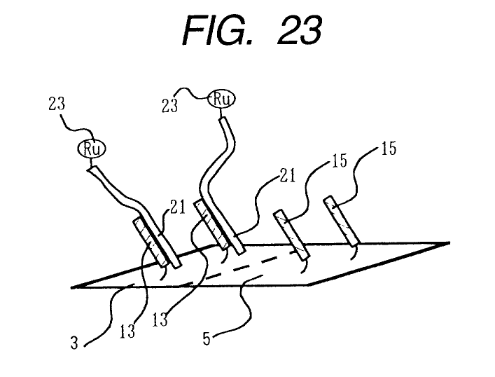

FIG. 23 illustrates a DNA probe hybridized with an ECL-labeled target polynucleotide. ECL is detected by the method described with reference to the first through sixth embodiments.

### 11th Preferred Embodiment

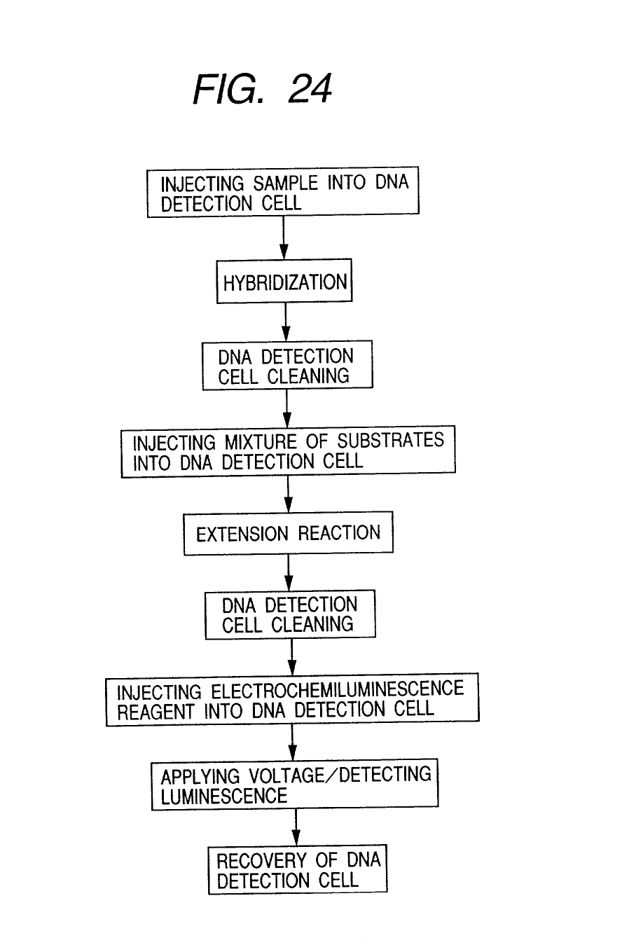

FIG. 24 illustrates the procedure of assaying with an assay apparatus. A sample solution containing the DNA fragment group to be measured is put into the DNA cell plate described with reference to different embodiments (for instance a cell formed of the DNA detecting cell base plate 11 and the upper DNA detecting cell plate 12). Then the temperature of the solution is set to be appropriate for hybridization, and the DNA probes and the DNA fragments are hybridized. The temperature at which the hybridization of the DNA probes and the DNA fragments is most efficiently accomplished and non-specific hybridization can hardly take place is determined in advance between 55 and 65° C. to be set for the solution. After the hybridizing reaction, DNA fragments not hybridized at normal temperature are discharged out of the DNA detecting cell using as the cleaning solution 20 mM phosphoric acid buffer solution (pH 7.0) to which 0.05% Tween 20 has been added.

Next, the extending reaction of the DNA probes hybridized with the target DNA fragments is carried out. After the extending reaction, the cell is washed with a cleaning solution to remove unreacted substrate, an ECL reagent is injected into the cell, and a voltage is applied between the working electrode and the counter electrode to detect the ECL that is generated. After the completion of ECL detection, and the DNA probe fixed to each luminous area of the DNA detecting cell is freed to regenerate the DNA detecting cell. The DNA detecting cell can be regenerated by any of the three typical methods described below.

By a first regeneration method, the DNA probe fixed to each luminous area of the DNA detecting cell is freed and completely removed, and a new DNA probe is fixed. The first method, as it leaves almost no sample, hardly invites false positivity.

By a second regeneration method, the DNA detecting cell is washed with pure water of 95° C. to free target DNA fragments from DNA probes, and only target DNA fragments are removed from the detecting cell. The second method can simply regenerate the DNA detecting cell in a short period of time, and is effective for the ninth and 10th embodiments.

A third regeneration method is effective for the seventh embodiment. By the third regeneration method, first the DNA detecting cell is washed with pure water of 95° C. to free target DNA fragments from DNA probes and removed from the DNA detecting cell. As a result, the DNA probes (15 in FIG. 7) and single chains of DNA probes having extended parts (26 and 27 in FIG. 7) resulting from hybridization remain in the DNA detecting cell. Next, when S1 nuclease is injected into the DNA detecting cell, the S1 nuclease decomposes the extended parts 26 and 27 into mononucleotides. The DNA probes, not decomposed by the S1 nuclease, remain fixed to the DNA detecting cell, which is regenerated to its state before use. The third regeneration method allows DNA probes to be reused, and can also decompose with the S1 nuclease the target DNA fragments which might otherwise remain in the DNA detecting cell, resulting in a characteristic of leaving no sample.

### Sequence Listing

Sequence number: 1

Length of sequence: 30

Pattern of sequence: Nucleic acid

Number of chain(s): One

Topology: Straight chain

Type of sequence: Other nucleic acid, synthetic DNA

Sequence: TCTCACACCAGCTGTCCCAAGACCGTTTGC

Sequence number: 2

Length of sequence: 30

Pattern of sequence: Nucleic acid

Number of chain(s): One

Topology: Straight chain

Type of sequence: Synthetic DNA

Sequence: AATACAGGCATCCTTCACTACATTTTCCCT

1. A polynucleotide assay apparatus characterized in that it has a polynucleotide detecting cell provided with a first electrode (111, 52, 60) to which different DNA probes (13, 14, 15, 16) are fixed in luminous areas (3, 4, 5, 6, 61-1 through 61-6, 82-1 through 82-4) differing with the type of DNA probe and a second electrode(s) (113-1, 113-2, 53, 62-1 through 62-3, 83-1 through 83-4) opposite to said first electrode; a voltage applying unit (44) for applying a voltage between said first electrode and said second electrode; and an optical detector (33, 34, 35, 36, 43, 72-1, 72-2, 246) for trapping said target polynucleotide through hybridization between said DNA probes fixed to said luminous areas and target polynucleotides (21), carrying out an extending reaction using a base (24) labeled with electrochemiluminescence (ECL) to extend said hybridized DNA probes, and thereby detecting ECL resulting from the application of said voltage; and the presence or absence of any extended chain (26) generated by said extending reaction is detected.

2. A polynucleotide assay apparatus, as stated in claim 1, characterized in that said ECL label is a ruthenium complex or an osmium complex.

3. A polynucleotide assay apparatus, as stated in claim 1, characterized in that said optical detector is a pickup device (43, 266) for detecting said ECL from a plurality of said luminous areas as a 2D image.

4. A polynucleotide assay apparatus, as stated in claim 1, characterized in that said second electrode is configured of a plurality of electrodes, said apparatus being provided with electrode selectors (62-1S through 62-3S, 91-1 through 91-4) for selecting a prescribed electrode out of said plurality of electrodes, and said voltage is applied between said electrode selected by said electrode selector and said first electrode to detect ECL from a prescribed luminous area selected out of said plurality of luminous areas.

5. A polynucleotide assay apparatus, as stated in claim 4, characterized in that said electrode selector is provided with TFT gate lines (91-1 through 91-4) each connected to one or another of said plurality of electrodes.

6. A polynucleotide assay apparatus, as stated in claim 1, characterized in that said first electrodes and said second electrodes are arranged on the same plane in alternate repetition in parallel in one direction, said apparatus having a device (45) for controlling the duration of the application of said voltage on the basis of the velocity of the expansion of the region in which said ECL occurs and the distance between the center line of said first electrodes arranged in alternate repetition in said one direction and the center line of said second electrode in said one direction.

7. A polynucleotide assay apparatus, as stated in claim 6, characterized in that said voltage is repeatedly applied.

8. A polynucleotide assay apparatus characterized in that it has a polynucleotide detecting cell provided with a first electrode (111, 52, 60) to which different DNA probes (13, 14, 15, 16) are fixed in luminous areas (3, 4, 5, 6, 61-1 through 61-6, 82-1 through 82-4) differing with the type of DNA probe and a second electrode(s) (113-1, 113-2, 53, 62-1 through 62-3, 83-1 through 83-4) opposite to said first electrode; a voltage applying unit (44) for applying a voltage between said first electrode and said second electrode; and an optical detector (33, 34, 35, 36, 43, 72-1, 72-2, 246) for trapping said target polynucleotide through hybridization between said DNA probes fixed to said luminous areas and target polynucleotides (21) to which is coupled oligonucleotide (28) labeled with ECL.

9. A polynucleotide assay apparatus characterized in that it has a polynucleotide detecting cell provided with a first electrode (111, 52, 60) to which different DNA probes (13, 14, 15, 16) are fixed in luminous areas (3, 4, 5, 6, 61-1 through 61-6, 82-1 through 82-4) differing with the type of DNA probe and a second electrode(s) (113-1, 113-2, 53, 62-1 through 62-3, 83-1 through 83-4) opposite to said first electrode; a voltage applying unit (44) for applying a voltage between said first electrode and said second electrode; and an optical detector (33, 34, 35, 36, 43, 72-1, 72-2, 246) for trapping said target polynucleotide through hybridization between said DNA probes fixed to said luminous areas and target polynucleotides (21) and detecting ECL resulting from the application of said voltage.

10. A polynucleotide assay apparatus characterized in that it has a polynucleotide detecting cell provided with a first electrode (111, 52, 60) to which different DNA probes (13, 14, 15, 16) are fixed in luminous areas (3, 4, 5, 6, 61-1 through 61-6, 82-1 through 82-4) differing with the type of DNA probe and a plurality of second electrodes (113-1, 113-2, 53, 62-1 through 62-3, 83-1 through 83-4) opposite to said first electrode; electrode selectors (62-1S through 62-3S, 91-1 through 91-4) for selecting an electrode out of said plurality of second electrodes; and a voltage applying unit (44) for applying a voltage between said first electrode and said selected electrode, wherein said target polynucleotides trapped through hybridization between target polynucleotides qualified with an ECL label and said DNA probes are detected for each luminous area selected out of said plurality of luminous areas by generating ECL from said ECL label by the application of said voltage.

11. A polynucleotide assay apparatus characterized in that it has a polynucleotide detecting cell provided with a first electrode (111, 52, 60) to which different DNA probes (13, 14, 15, 16) are fixed in luminous areas (3, 4, 5, 6, 61-1 through 61-6, 82-1 through 82-4) differing with the type of DNA probe and a plurality of second electrodes (83-1 through 83-4) arranged on the same plane as said first electrode, separated from said first electrode and each arranged in the central part of one or another of said luminous areas; electrode selectors (91-1 through 91-4) for selecting an electrode out of said plurality of second electrodes; a voltage applying unit (44) for applying a voltage between said first electrode and said selected electrode; and an optical detector (33, 34, 35, 36, 43, 72-1, 72-2, 246) for detecting ECL generated from the ECL label by the application of said voltage, further having a device (45) for controlling the duration of the application of said voltage on the basis of the distance between the central part of said selected second electrode and the boundary of said luminous area adjoining said luminous area in which said selected second electrode is arranged and the velocity of the expansion of the region in which said ECL occurs; wherein said target polynucleotide trapped in each of said luminous areas is detected.

12. A polynucleotide assay apparatus, as stated in claim 11, characterized in that said plurality of second electrodes are arranged at equal intervals in two directions.

13. A polynucleotide assay apparatus characterized in that it has a polynucleotide detecting cell provided with a first electrode (111, 52, 60) to which different DNA probes (13, 14, 15, 16) are fixed in luminous areas (3, 4, 5, 6, 61-1 through 61-6, 82-1 through 82-4) differing with the type of DNA probe and a plurality of second electrodes (53, 62-1 through 62-3, 83-1 through 83-4) arranged on the same plane as said first electrode; electrode selectors (62-1S through 62-3S, 91-1 through 91-4) for selecting an electrode out of said plurality of second electrodes; a voltage applying unit (44) for applying a voltage between said first electrode and said selected electrode; an optical detector (33, 34, 35, 36, 43, 72-1, 72-2, 246) for detecting ECL generated from the ECL label by the application of said voltage; and a device (45) for controlling the duration of the application of said voltage on the basis of the velocity of the expansion of the region in which said ECL occurs; wherein said target polynucleotide trapped in each of said luminous areas is detected.

14. A polynucleotide assay apparatus characterized in that it has a polynucleotide detecting cell provided with a first electrode (111, 52, 60) to which different DNA probes (13, 14, 15, 16) are fixed in luminous areas (3, 4, 5, 6, 61-1 through 61-6, 82-1 through 82-4) differing with the type of DNA probe and a plurality of second electrodes (113-1, 113-2, 53, 62-1 through 62-3, 83-1 through 83-4) opposite to said first electrode; electrode selectors (62-1S through 62-3S, 91-1 through 91-4) for selecting an electrode out of said plurality of second electrodes; and a voltage applying unit (44) for applying a voltage between said first electrode and said selected electrode, wherein said target polynucleotide trapped in each of said luminous areas is detected by detecting for each luminous area selected out of said plurality of luminous areas by generating ECL from said ECL label by the application of said voltage.

15. A polynucleotide assay method characterized in that it has a step to trap target polynucleotides by hybridizing DNA probes fixed to luminous areas in a polynucleotide detecting cell, which is provided with a first electrode (111, 52, 60) in which different DNA probes (13, 14, 15, 16) are fixed to luminous areas (3, 4, 5, 6, 61-1 through 61-6, 82-1 through 82-4) differing with the type of DNA probe and a second electrode(s) (113-1, 113-2, 53, 62-1 through 62-3, 83-1 through 83-4) opposite to said first electrode, with target polynucleotides (21); a step to subject said hybridized DNA probes to an extending reaction using an ECL-labeled base (24) to extend said DNA probes; a step to apply a voltage between said first electrode and said second electrode(s); and a step to detect the presence or absence of any extended chain (26) generated by said extending reaction by detecting the presence or absence of ECL resulting from the application of said voltage.

16. A polynucleotide assay method characterized in that it has a step to trap target polynucleotides by hybridizing DNA probes (13, 14, 15, 16) fixed to luminous areas (3, 4, 5, 6, 61-1 through 61-6, 82-1 through 82-4) differing with the type of DNA probe in a polynucleotide detecting cell, which is provided with a first electrode (111, 52, 60) and a second electrode(s) (113-1, 113-2, 53, 62-1 through 62-3, 83-1 through 83-4) opposite to the first electrode, with the target polynucleotides (21) to each of which an ECL-labeled oligonucleotide (28) is coupled; and a step to apply a voltage between said first electrode and said second electrode(s) and thereby detect any ECL resulting from the application of said voltage.

17. A polynucleotide assay method characterized in that it has a step to trap target polynucleotides by hybridizing DNA probes (13, 14, 15, 16) fixed to luminous areas (3, 4, 5, 6, 61-1 through 61-6, 82-1 through 82-4) differing with the type of DNA probe in a polynucleotide detecting cell, which is provided with a first electrode (111, 52, 60) and a second electrode(s) (113-1, 113-2, 53, 62-1 through 62-3, 83-1 through 83-4) opposite to the first electrode, with the ECL-labeled target polynucleotides (21); and a step to apply a voltage between said first electrode and said second electrode(s) and thereby detect any ECL resulting from the application of said voltage.

18. A polynucleotide assay method characterized in that it has a step to select, in a polynucleotide detecting cell provided with a first electrode (111, 52, 60) to which different DNA probes (13, 14, 15, 16) are fixed in luminous areas (3, 4, 5, 6, 61-1 through 61-6, 82-1 through 82-4) differing with the type of DNA probe and a plurality of second electrodes (53, 62-1 through 62-3, 83-1 through 83-4) arranged on the same plane as said first electrode, an electrode out of said plurality of second electrodes; a step to apply a voltage between said first electrode and said selected electrode; a step to detect any ECL generated from an ECL label by the application of said voltage; and a step to control the length of time during which said voltage is applied and held on the basis of the velocity of the expansion of the region in which said ECL occurs; wherein said target polynucleotide trapped in each of said luminous areas is detected.

19. A polynucleotide assay method, as stated in claim 18, characterized in that said voltage is applied and held for a length of time substantially equal to the length of time required by the expansion of the region in which said ECL occurs to reach said luminous area adjoining said luminous area in which a selected electrode out of said plurality of second electrodes is arranged.
